SEQUENCE LISTING (13 sequences)

SEQ ID NO: 1 - DNA, 20 bp, Artificial Sequence
  primer
ctsatcatcwykgtstgyta

SEQ ID NO: 2 - DNA, 25 bp, Artificial Sequence
  primer
gcggatccttgaagttytcrtccag

SEQ ID NO: 3 - DNA, 20 bp, Artificial Sequence
  primer
gcgcctccgtgtacttctaa

SEQ ID NO: 4 - DNA, 20 bp, Artificial Sequence
  primer
gatggcagcctctaagttta

SEQ ID NO: 5 - DNA, 20 bp, Artificial Sequence
  primer
aaccatggacagcagcgccg

SEQ ID NO: 6 - DNA, 20 bp, Artificial Sequence
  primer
gccactagcacgctgccctt

SEQ ID NO: 7 - DNA, 25 bp, Artificial Sequence
  primer
cagtggatcgaactaaccaccagct

SEQ ID NO: 8 - DNA, 25 bp, Artificial Sequence
  primer
ggattttgctcagaatggtggcatg

SEQ ID NO: 9 - DNA, 25 bp, Artificial Sequence
  primer
aatgcattcttgctcctcaaggatc

SEQ ID NO: 10 - DNA, 25 bp, Artificial Sequence
  primer
tccctgggccggcgctgctgtccat

SEQ ID NO: 11 - DNA, 25 bp, Artificial Sequence
  primer
agtgggggcacatgaaacaggcttc

SEQ ID NO: 12 - DNA, 25 bp, Artificial Sequence
  primer
gagggttattaatgttgtcctttac

SEQ ID NO: 13 - DNA, 25 bp, Artificial Sequence
  primer
gttgttacaaagaaacttagagtct

(19) United States
(12) Patent Application Publication
(10) Pub. No.: US 2003/0003050 A1
(43) Pub. Date: Jan. 2, 2003

(54) Method for evaluating drug sensitivity

(76) Inventors: Kazutaka Ikeda, Wako-shi (JP); Hiroaki Niki, Wako-shi (JP); Ryoji Yano, Wako-shi (JP); Toshiro Kumanishi, Niigata-shi (JP); Toru Kobayashi, Niigata-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/073,647

(22) Filed: Feb. 11, 2002

(30) Foreign Application Priority Data
Jun. 13, 2001 (JP) 2001-178169

(51) Int. Cl. A61K 49/00; C12Q 1/68

(52) U.S. Cl. 424/9.1; 435/6

(57) ABSTRACT

A method for evaluating sensitivity of a human or animal to a drug, which comprises detecting a difference in an untranslated region of mRNA for a gene in which diversity in the untranslated region of mRNA affects the sensitivity to a drug, and evaluating the sensitivity to a drug based on the detected difference.

### BACKGROUND OF THE INVENTION

The present invention relates to a method for evaluating sensitivity of a human or animal to a drug.

It is known that there are individual differences in sensitivity to a drug such as analgesics and carcinostatics (drug sensitivity). In case of a drug showing strong side effects, in particular, administration thereof in an excess quantity invites serious results, and therefore it is desired to predict such individual differences. For this reason, the cause of the presence of individual differences in sensitivity to drugs has been studied, and it has been reported that diversity in a translated region of a gene affects on drug sensitivity.

However, there are genes not showing diversity in coding regions of the genes among genes involved in drug sensitivity, and it is difficult to evaluate drug sensitivity in which such genes participate, without actually administering a drug of interest. In case of morphine, for example, previous studies have revealed that intracerebral target of morphine is μ-opioid receptor. However, since this receptor shows almost no individual differences in protein structure, it is impossible to predict individual differences of analgesic effect of morphine based on the protein structure.

### SUMMARY OF THE INVENTION

An object of the present invention is to provide a method for evaluating drug sensitivity based on a novel parameter without administering a drug.

The inventors of the present invention investigated by using mice of CXBK strain, which is a mouse strain showing reduced analgesia by morphine. As a result, they found that diversity in untranslated region of mRNA significantly affected drug sensitivity. The present invention was accomplished based on this finding.

The present invention provides a method for evaluating sensitivity of a human or animal to a drug, which comprises detecting a difference in an untranslated region of mRNA for a gene in which diversity in the untranslated region of mRNA affects the sensitivity to a drug, and evaluating the sensitivity to a drug based on the detected difference.

The difference of the untranslated region may be a difference in length of the untranslated region.

As the gene, the μ-opioid receptor gene can be mentioned. In this case, the drug is a drug of which target is the μ-opioid receptor. When the gene is the μ-opioid receptor gene, morphine can be mentioned as the drug.

According to the present invention, it becomes possible to evaluate sensitivity to a drug without administering the drug, and administration of the drug becomes possible with taking individual difference in sensitivity to the drug into consideration. According to the evaluation method of the present invention, a suitable drug prescription can be recommended only by analyzing size, nucleotide sequence or the like of the untranslated region.

### BRIEF EXPLANATION OF THE DRAWING

FIG. 1 shows evaluation results for sensitivity to morphine and (−)-U-50488.

### DETAILED DESCRIPTION OF THE INVENTION

The evaluation method of the present invention is a method for evaluating drug sensitivity of a human or animal, and it is characterized by detecting a difference in an untranslated region of mRNA for a gene in which diversity in the untranslated region of mRNA affects the sensitivity to a drug, and evaluating the sensitivity to a drug based on the detected difference.

In the present invention, the drug is not particularly limited, so long as it is a drug acting on humans or animals. Examples thereof include analgesics, carcinostatics, anti-allergy agents, hypotensors, diuretics, anesthetics and so forth. The drug is preferably, in particular, one showing a large difference in sensitivity among individuals of human or animal. This is because a drug showing larger individual difference in sensitivity provides more significant influence when it is administered in an excess quantity to an individual having high sensitivity.

While the animal is not particularly limited, it is usually a vertebrate, preferably a mammal.

The gene may be any gene in which diversity in an untranslated region of mRNA affects drug sensitivity. The gene in which diversity in an untranslated region of mRNA affects drug sensitivity means a gene of which product affects drug sensitivity of an individual and which has a difference in a nucleotide sequence of its untranslated region among individuals showing difference in drug sensitivity (hereinafter also referred to as “a gene involved in drug sensitivity”). Examples of the gene involved in drug sensitivity include genes of drug receptors, genes of enzymes involved in metabolism of drugs and so forth.

The gene in which diversity in the mRNA untranslated region affects drug sensitivity can be found by such methods as mentioned below.

(1) For humans or animals showing difference in drug sensitivity, untranslated regions of the gene involved in drug sensitivity are amplified by PCR utilizing genomic DNA as a template or the like. Size or nucleotide sequence of the regions is analyzed by electrophoresis or sequencing method to identify a difference. A gene for which difference has found is a gene that can be used for the evaluation method of the present invention.

(2) mRNAs are prepared from humans or animals showing difference in drug sensitivity, and gene expression profiling is performed by using the mRNAs as probes based on the microarray method that enables simultaneous analysis of expression patterns for multiple genes. For a gene showing difference in expression, size or nucleotide sequence of its untranslated region is analyzed to identify the difference of the region and confirm its involvement in drug sensitivity. A gene for which a difference is identified and an involvement in drug sensitivity is confirmed, is a gene that can be used for the evaluation method of the present invention.

A gene that can be used for the evaluation method of the present invention is not limited to those found by the aforementioned methods, so long as it is a gene in which diversity in the mRNA untranslated region affects drug sensitivity.

As for the difference in the untranslated region, difference in nucleotide sequence may be detected, or when the difference in nucleotide sequence is reflected in size (length), it may be detected as a difference in length.

The method for detecting difference in an mRNA untranslated region is not particularly limited. For example, in case of difference in length, since the length of untranslated region is reflected in the full length of mRNA, the full length of mRNA may be measured. For example, it is possible to detect a difference in the length of mRNA by preparing mRNA from a sample, being subjected it to agarose gel electrophoresis and then performing Northern blot analysis with a labeled probe having a nucleotide sequence complementary to the mRNA. When nucleotide sequences of mRNAs having different lengths are each already elucidated or difference in nucleotide sequences is already elucidated, it is possible to detect the difference in the length or nucleotide sequence of mRNA by performing PCR amplification with primers designed based on the sequences so that the difference in the length or nucleotide sequences of mRNA should be reflected in a property (length etc.) of the amplified product, and cDNA prepared from a sample as a template, and investigating the property or presence or absence of the property of the amplified product. Further, when there has been elucidated a nucleotide sequence in genomic DNA of a gene that produces mRNAs having different lengths or mRNAs having different nucleotide sequences, it is possible to detect the difference in the length or nucleotide sequence of mRNA by performing PCR amplification with primers designed based on the nucleotide sequence so that the difference in the length of mRNA or difference in the nucleotide sequences should be reflected in a property (length etc.) of the amplified product, and cDNA or genomic DNA prepared from a sample as a template, and investigating the property or presence or absence of the property of the amplified product.

As an example of the gene in which diversity of the untranslated region affects drug sensitivity, a μ-opioid receptor gene can be mentioned. The untranslated region of the μ-opioid receptor gene shows a difference that is reflected in a difference in length of the untranslated region.

When the μ-opioid receptor gene is used as a gene involved in drug sensitivity, the drug may be a drug of which target is the μ-opioid receptor, and an example of such a drug is morphine.

The reason why drug sensitivity can be evaluated by the method of the present invention may be considered as follows. As shown in the examples described below, it was found that, in CXBK mice, which are known to show reduced analgesic effect of morphine, the untranslated region of the μ-opioid receptor gene was abnormally long, the intracerebral quantity of mRNA of the μ-opioid receptor gene was decreased, and the coded μ-opioid receptor itself did not show abnormality. Furthermore, it was confirmed that the abnormality of the mRNA untranslated region of the μ-opioid receptor gene correlated with the reduction of the morphine analgesic effect. From the above, it was found that the sensitivity to morphine could be evaluated based on the nucleotide sequence of the mRNA untranslated region of the μ-opioid receptor gene. It is predicted that such evaluation is possible because mRNA becomes unstable due to the abnormality of the mRNA untranslated region to invite its reduced intracerebral quantity, and as a result, the intracerebral quantity of the μ-opioid receptor is decreased so that the analgesic effect by morphine is reduced. It is also considered that sensitivity to a drug of which target is the μ-opioid receptor (opioid) can be similarly evaluated like the sensitivity to morphine.

It is considered that, since the nucleotide sequence of the untranslated region is not conserved so much in an evolutional process, the mRNA untranslated region of the μ-opioid receptor gene should be diverse among individuals not only in mouse but also in other animals or human. Therefore, it is considered similarly possible in animals other than mouse or human that the untranslated region significantly affects the stability of mRNA to cause individual difference in the amount of mRNA, and the size of mRNA corresponds to the amount of the μ-opioid receptor protein to cause individual difference in the protein amount and eventually cause individual difference in the effect of an opioid. Furthermore, it is also considered that, not limited to the μ-opioid receptor gene, mRNA untranslated region is considered to be diverse among individuals in other genes involved in drug sensitivity.

### EXAMPLES

Hereafter, the present invention will be explained with reference to the following examples.

### Example 1

(1) Abnormal μ-opioid Receptor (μ-OR) mRNA in CXBK Mice

To investigate the expression of opioid receptor (OR) mRNAs in CXBK mice, Northern blot analyses were conducted.

The mice were housed in an aluminum cage with littermates of the same sex (up to five per cage) in an environment maintained at 23±1° C. and a relative humidity of 50±5% with a 12-hour light/dark cycle (lights on 7:00 A.M. to 7:00 P.M.). The mice had access to a standard commercial laboratory diet ad libitum (NMF; Oriental Yeast Co. Ltd.) and water. The CXBK mice were originally purchased from The Jackson Laboratory. C57BL/6CrSlc (B6) and BALB/cCrSlc (BALB/c) mice were purchased from Japan SLC. The experimental procedures and housing conditions were approved by the Institutional Animal Care and Use Committee. All of the animals were cared for and treated humanely, in accordance with the animal experimentation guidelines of the present inventors' institution.

mRNAs were separately prepared from the brain of each naive adult male mouse by using Messenger RMA Isolation kit (Stratagene). RNA size markers were purchased from Novagen. The RNAs were electrophoresed on 1% agarose gel containing formaldehyde and transferred to a nitrocellulose membrane (PROTRAN; Schleicher & Schuell) or a nylon membrane (Hybrid-N+; Amersham Pharmacia Biotech). The probes for μ-, δ- and K-opioid receptor mRNAs were prepared by PCR with Pfu DNA polymerase (Stratagene), pSPORμ, pSPORδ and pSPORκ as the templates, respectively. The common pair of primers for fragments corresponding to the transmembrane V-VII regions of the receptors were 5′-CT(C/G)ATCATC(A/T)(C/T)(G/T)GT(C/G)TG(C/T)TA-3′ (sense primer: SEQ ID NO: 1) and 5′-GCGGATCCTTGAAGTT(C/T)TC(C/G)TCCAG-3′ (antisense primer: SEQ ID NO: 2). The hybridization was performed at 60° C. for 20 hours in a hybridization solution (ExpressHyb Hybridization Solution; Clonetech) with [³²P]-labeled probe (2×10⁶ cpm/ml). The blots were washed at 42° C. in 0.1× SSC (150 mM NaCl and 25 mM sodium citrate) containing 0.1% SDS. Autoradiograpy was performed and analyzed by using BAS-5000 Imaging Analyzer (Fujifilm). The values of photostimulated luminescence (PSL), which are proportional to the radioactivity in arbitrary measured areas (Amemiya et al., Science, 237:164-168, 1987) were compared in quantitative analyses. The membranes were dehybridized in 0.1× SSC solution containing 0.1% SDS at 100° C. for 10 minutes. Expressions of μ-, δ- and κ-OR mRNAs were analyzed using the same membranes.

As a result, it was found that the CXBK mice had a large-sized (14.5 kb) μ-OR mRNA in their brains whereas the progenitor strain of mice, B6 mice, had 12 kb μ-OR mRNA. The heterozygotes between B6 and CXBK mice had both mRNAs, although the signal for the 14.5 kb mRNA was faint. The other progenitor stain of mice, BALB/c mice, had only 12 kb μ-OR mRNA. The signal intensity for μ-OR mRNA in CXBK mice was reduced to about 60% of the intensity in B6 and BALB/c mice, when equal amounts of brain mRNAs were electrophoresed and analyzed. Although the size of δ-OR mRNA was the same in all strains, the signal intensity for δ-OR mRNA in CXBK mice was higher than that in B6 and BALB/c mice. The size of κ-OR mRNA and the signal intensity for the mRNA in all strains were not significantly different. The size difference in μ-OR mRNA suggests that the μ-OR gene in CXBK mice may be different from that of the progenitor strain mice.

(2) Distribution of μ-OR mRMA in CXBK Mice

By using in situ hybridization histochemistry, the expression of the μ-OR mRNA in the CXBK mouse brains was compared with that in the B6 mouse brains.

The probe for μ-OR mRNA was a 45-mer oligonucleotide complementary to a part of a μ-OR cDNA sequence, including the initial methionine codon (Ikeda et al., Ann. NY Acad. Sci., 801:95-109, 1996). The oligonucleotide was labeled with [³³P]-dATP using terminal deoxyribonucleotidyl transferase (Takara Shuzo) and purified by using a Sephadex G-25 Spin Column (Boehringer Mannheim). The specific activity of the probe was 5×10⁸ dpm/μg. In situ hybridization histochemistry was performed as described previously (Ikeda et al., J. Comp. Neurol., 399:139-151, 1998). Horizontal and sagittal sections of adult male B6 and CXBK mouse brains were placed on slides and fixed with 4% paraformaldehyde/0.1 M sodium PBS. The sections were hybridized in a hybridization solution containing 5×10³ dpm/μl probe for 16 hours at 42° C. The slides were washed three times in 0.1× SSC −0.1% Sarkosyl at 55° C. for 40 minutes for each washing, dehydrated and analyzed by using BAS-5000 Imaging Analyzer (Fujifilm). Values of PSL were compared by quantitative analyses. Afterward, the slides were exposed to Hyperfilm-β-max (Amersham Pharmacia Biotech) for 2 weeks to obtain X-ray film images.

As a result, in the CXBK mouse brain, the μ-OR mRNA was expressed in a variety of brain regions in a similar manner to the B6 mouse brain. However, the signal intensity for the mRNA in the CXBK mouse brain was significantly lower (about 70% of that in the B6 mouse brain), which was consistent with the results of the Northern blot analyses. Similar results were obtained by using sagittal sections of the B6 and CXBK mouse brains. These results suggest that the expression level of the μ-OR mRNA was homogeneously lower in the CXBK mouse brains.

(3) Nucleotide Difference between B6 and CXBK Mouse μ-OR Genes

A part (2184 bases; GenBank accession number AB047546) of the μ-OR mRNA, including the entire coding region, was compared in B6 and CXBK mice. Sequencing was performed as follows.

The CXBK and B6 mouse brain cDNAs were synthesized with the corresponding mRNAs as the templates by using 1st Strand cDNA Synthesis kit (Clontech). Genomic DNAs were prepared from mouse tail or liver. DNA fragments were amplified by PCR with Pfu DNA polymerase. The PCR primers for μ-OR cDNA were 5′-GCGCCTCCGTGTACTTCTAA-3′ (sense primer: SEQ ID NO: 3) and 5′-GATGGCAGCCTCTAAGTTTA-3′ (antisense primer: SEQ ID NO: 4). The nucleotide sequence of the PCR product was analyzed with the PCR primers and other primers as follows: 5′-AACCATGGACAGCAGCGCCG-3′ (SEQ ID NO: 5), 5′-GCCACTAGCACGCTGCCCTT-3′ (SEQ ID NO: 6), 5′-CAGTGGATCGAACTAACCACCAGCT-3′ (SEQ ID NO: 7) and 5′-GGATTTTGCTCAGAATGGTGGCATG-3′ (SEQ ID NO: 8, Kaufman et al., J. Biol. Chem., 270:15877-15883, 1995). The PCR primers for the μ-OR genes (5′-flanking region to the translation starting site) were 5′-AATGCATTCTTGCTCCTCAAGGATC-3′ (sense primer: SEQ ID NO: 9) and 5′-TCCCTGGGCCGGCGCTGCTGTCCAT-3′ (antisense primer: SEQ ID NO: 10). The nucleotide sequence of the PCR product was analyzed with the PCR primers and other primers as follows: 5′-AGTGGGGGCACATGAAACAGGCTTC-3′ (SEQ ID NO: 11), 5′-GAGGGTTATTAATGTTGTCCTTTAC-3′ (SEQ ID NO: 12) and 5′-GTTGTTACAAAGAAACTTAGAGTCT-3′ (SEQ ID NO: 13, Liang et al., Brain Res., 679:82-88, 1995). The nucleotide sequencing was conducted by using PRISM 310 genetic analyzer (Applied Biosystems).

As a result, it was found that the sequence of the coding region (1197 bases) of the μ-OR mRNA in CXBK mice was identical to that in the B6 mice, indicating that the μ-OR protein structure is normal, but the untranslated region (UTR) of the μ-OR mRNA is abnormally long in CXBK mice. A sequence difference was not apparent in the examined 3′-UTR (726 bases), and there was only a single nucleotide sequence difference in the examined 5′-UTR (214 bases). This indicated that the difference in the size of the μ-OR mRNA between the B6 and the CXBK mice would be in the unexamined UTR of the μ-OR mRNA. The 5′-flanking region (1107 base pairs; GenBank accession number AB047547) of the translation starting site was also compared for the B6 and CXBK μ-OR genes. A sequence difference between them was not detected except that corresponding to the difference in the 5′-UTR. It was unlikely that the single nucleotide sequence difference caused whole CXBK phenotypes, because BALB/c mice possessed the same nucleotide sequence in this region as CXBK mice.

(4) Mice Inheriting Two Copies of the CXBK μ-OR Gene (CXμ)

To understand the correlation between the CXBK μ-OR gene and the CXBK phenotypes, littermates were prepared by mating heterozygotes between B6 and CXBK mice. These littermates were as follows: mice inheriting two copies of the B6 μ-OR gene (B6p), mice inheriting two copies of the CXBK μ-OR gene (CXμ), and mice inheriting one copy of the B6 μ-OR gene and one copy of the CXBK μ-OR gene (Heμ). By using these littermates, Northern blot analyses was conducted in the same manner as in (1) to clarify whether the differences in the size and amount of OR mRNAs in the CXBK mouse brains were attributable to the CXBK μ-OR gene. As a result, it was assumed that the sizes of the μ-OR mRNAs in B6μ and CXμ mice were estimated to be the same as the B6 and the CXBK mice, respectively. The signal intensities for the μ- and δ-OR mRNAs in CXμ mice were low and high, respectively, when compared with the signal intensities in B6μ mice. Hep mice possessed both of these μ-OR mRNAs in a similar manner to the heterozygotes between B6 and CXBK mice. These results suggest that the CXBK μ-OR gene caused the differences in the size and expression levels of the OR mRNAs in the CXBK mice.

(5) Reduced Sensitivity to Opioid of CXμ Mice

Second, using these littermates, whether the CXBK μ-OR gene is associated with reduction of morphine effects in CXBK mice was investigated by using tail-flick test, hot-plate test and open-field test. These tests were conducted as follows.

Naive adult (6- to 15-week old) mice were used in all the experiments. Each mouse was tested in the daytime (not earlier than 8:00 A.M. and not later than 5:00 P.M). After the mouse was weighed, the tail-flick, open-field and hot-plate tests were performed (in that sequence) to examine the basal reactivities and activity. Morphine hydrochloride (10 mg/ml) was purchased from Takeda Chemical Industries, Ltd. (1S-trans)-3,4-dichloro-N-methyl-N-(2-[1-pyrrolidinyl]cyclohexyl)-benzeneacetamide hydrochloride [(−)-U-50488] (Research Biochemicals) was dissolved in distilled water, and the stock solution was stored at −20° C. until used. Each drug solution was diluted to 1 mg/ml with sterilized saline (0.9% NaCl) on each experimental day. The drug solution was injected intraperitoneally to the mouse at a dose of 10 ml/kg. The tail-flick, open-field and hot-plate tests were performed 10, 15 and 20 minutes after the injection, respectively. The tail-flick test was performed according to the method of D'Amour and Smith (J. Pharmacol. Exp. Ther., 72:74-79, 1941) with slight modification (Ikeda et al., Neurosci. Res., 34:149-155, 1999). The cutoff time was 15 seconds. The hot-plate test was performed according to the method of Woolf and MacDonald (J. Pharmacol. Exp. Ther., 80:300-307, 1944) with a slight modification (Ikeda et al., Neurosci. Res., 34:149-155, 1999). The temperature of the metal plate was adjusted to 52.0±0.2° C. The latency, from the test start to the first jumping, was measured, and the cutoff time was 300 seconds. The open-field test was performed as described previously (Ikeda et al., Mol. Brain Res., 33:61-71, 1995). The horizontal and vertical locomotions of the mouse were measured for 300 seconds. In this experiment, because the various kinds of locomotions were well correlated, the walking distance was used as the mouse locomotion. An ANOVA and Scheffe's F post hoc test were used to statistically analyze the between group data, with p<0.05 accepted as statistically significant.

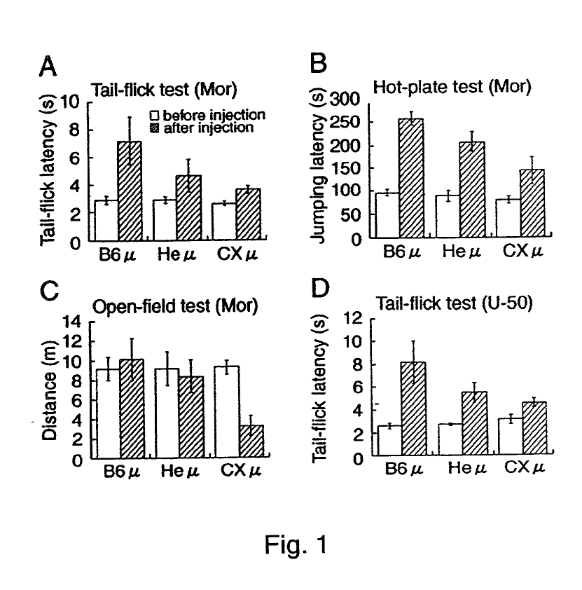

The results of the tail-flick and hot-plate tests performed for morphine (Mor) induced analgesic effect are shown in FIGS. 1, A and B, and the results of the open-field test performed for morphine-induced hyperactivity are shown in FIG. 1, C. Further, the result of the investigation for whether the CXBK μ-OR gene induces reduction of analgesic effects of (−)-U-50488 (U-50), which is a selective κ-agonist, in CXBK mice are shown in FIG. 1, D. The values mentioned in FIG. 1 are represented as average +SEM. Each group consisted of 10 animals for A to C, and each group consisted of 6 animals for D.

The B6μ, Heμ and CXμ mice responded to the heat stimuli with similar latencies and showed similar spontaneous activity when they were not given morphine. However, after intraperitoneal administration of 10 mg/kg morphine, CXμ mice responded to heat stimuli with a significantly shorter latency than the littermates in both analgesic tests (p<0.05; repeated-measure ANOVA), indicating that the CXμ mice showed lower morphine-induced analgesia. In the open-field test, B6μ and Heμ mice walked similar distances before and after morphine administration, indicating that the decrease in locomotor activity attributable to habituation was counterbalanced by morphine-induced hyperactivity in these mice. In contrast, CXμ mice walked significantly shorter distances after morphine administration than they did before morphine administration (p<0.001; paired t test), indicating that morphine failed to counterbalance the inhibiting effects of habituation on the locomotion of CXμ mice. These results suggested that the reduced effects of morphine on analgesia and locomotion in CXBK mice were correlated with the CXBK μ-OR gene.

In the tail-flick test, the CXμ mice responded to the heat stimulus with a significantly shorter latency than the littermates after intraperitoneal administration of 10 mg/kg (−)-U-50488 (p<0.05; repeated measure ANOVA). This result suggests that the reduction of (−)-U-50488-induced analgesia in the CXBK mice was also associated with the CXBK μ-OR gene.

These three correlations between the CXBK μ-OR gene and the CXBK phenotypes suggest that the CXBK μ-OR gene contributed to the CXBK phenotypes.

What is claimed is:

1. A method for evaluating sensitivity of a human or animal to a drug, which comprises detecting a difference in an untranslated region of mRNA for a gene in which diversity in the untranslated region of mRNA affects the sensitivity to a drug, and evaluating the sensitivity to a drug based on the detected difference.

2. The method according to claim 1, wherein the difference of the untranslated region is a difference in length of the untranslated region.

3. The method according to claim 1, wherein the gene is μ-opioid receptor gene and the drug is a drug of which target is the μ-opioid receptor.

4. The method according to claim 2, wherein the gene is μ-opioid receptor gene and the drug is a drug of which target is the μ-opioid receptor.

5. The method according to claim 3, wherein the drug is morphine.

6. The method according to claim 4, wherein the drug is morphine.
